(19) United States
(12) Patent Application Publication
(10) Pub. No.: US 2016/0262903 A1
(43) Pub. Date: Sep. 15, 2016

(54) TALAR DOME PROSTHESIS

(71) Applicant: WRIGHT MEDICAL TECHNOLOGY, Memphis, TN (US)

(72) Inventor: Jerry W. WEST, Arlington, TN (US)

(21) Appl. No.: 14/402,622

(22) PCT Filed: Sep. 12, 2014
(86) PCT No.: PCT/US14/55294

§ 371 Date: Nov. 20, 2014

(51) Int. Cl. A61F 2/42

(52) U.S. Cl. CPC A61F 2/4202; A61F 2002/4207; A61F 2002/3065

(57) ABSTRACT

A talar dome is provided as an element of a total ankle arthroplasty, to fit over a resected talus bone. The talar dome has an integral dome body with a smooth and rounded superior articulating side facing the tibia, and an inferior mounting side with mutually inclined flat faces that complement the flat surfaces of the resected talus. On the inferior or mounting side, the talar dome has at least two pegs integral with and rigidly protruding from the dome body. The pegs each taper along flat faces to a point that is embedded in the talus bone for operatively attaching the talar dome to the talus bone. At least one flat face is of each peg is perpendicular to the sagittal plane and resists anterior/posterior displacement of the talar dome until healed.

### FIELD OF THE INVENTION

This disclosure relates to the field of ankle arthroplasty including methods and apparatus for supplanting the surface of the talus with a prosthetic implant adapted to cooperate with a tibial prosthesis.

### BACKGROUND

US published application 2012/0271314-Stemniski et al., hereby incorporated by reference in its entirety, discloses aspects of total ankle replacement arthroplasty based on the coordinated use of a preoperative alignment fixture, several associated tool guides coupled to the fixture that conform the motion of surgical tools used during a surgical procedure, and prosthetic members that are installed to terminate the distal tibia and to engage over the head of the talus, articulating with one another as a prosthetic ankle joint.

More particularly, the fixture is preoperatively adjusted to conform to the patient's anatomy while fluoroscopically viewing the tibia and aligning the fixture. The distal tibia and the superior talus are resected using a bone saw applied through an anterior incision. The saw cutting path is guided along slots in the fixture while aligned to the patient's anatomy. Three linear saw cuts in the tibia separate a trapezoidal piece of bone that is removed to leave a mortise in the distal tibia, accurately matched to the size and shape of a tibial plate prosthesis that will be the proximal part of a prosthetic ankle joint. Plural lateral cuts at different inclination angles resect the dome of the talus to leave the talus faceted along surfaces that accurately match surfaces on an underside of a talar dome prosthesis.

Certain bore holes are drilled, likewise guided by the fixture, to receive stabilizing posts or other elongated fasteners that engage with the tibial plate and/or talar dome prostheses. If the stabilizing posts and bore holes for one or another of the prostheses are parallel and there is sufficient clearance available, the posts can be fixedly attached to the prosthesis or integral with the prosthesis, and inserted into their associated bore holes when placing the prosthesis.

In some surgical procedures and embodiments, an elongated post for the tibial prosthesis is to extend into the cancellous axial part of the distal tibia occupies a substantial diameter as an intramedullary supporting structure. There is little clearance for this aspect, but a bore for an intramedullary supporting structure can be formed via a plantar incision, drilled through the talus and into the cancellous axial part of the distal tibia, once again while precisely guided by the fixture. Anterior access through saw-cut mortise permits the tibial bore to be reamed. An intramedullary post structure is built and inserted into the tibia in axially short segments that attach to one another.

Embodiments of the fixture and technique are used in the Wright Medical Technology, Inc. PROPHECY® preoperative navigation alignment guides, and the INBONE® and INFINITY® total ankle systems. The INBONE and INFINITY systems each require supporting posts affixed to the talar dome and extending into post holes that are bored and reamed in the talus. The supporting posts are surfaced with a porous metal coating such as Wright Medial Technology BIOFOAM®, a sintered titanium alloy material whose rough and porous surface enhances bone ingrowth during healing.

### SUMMARY

An object of this disclosure is to provide an ankle arthroplasty talar dome implant functionally replaces the rounded top of the talus bone in a manner that is similar to the function of the talar dome prostheses mentioned above, but is easier to manufacture, easier to install and correspondingly effective in a total ankle arthroplasty. The implant has a rounded articulating dome on its upper (superior) side for bearing against a tibial plate structure as the opposed member of a prosthetic ankle joint. The implant has plural angled faces on an underside, for complementary abutment in surface contact with surfaces of a resected talus. In one embodiment, three flat faces are provided on the underside, of which the anterior and posterior faces are oppositely inclined toward one another, for example at about 20° relative to a horizontal central face, forming a partial enclosure over the talar dome. Preferably, this partial enclosure covers over the top of the talus but does not include lateral and medial sidewall flanges.

According to one aspect, the implant includes plural affixation pegs, preferably integral with the cast surgical alloy of the talar dome, such as austenitic 316 stainless and martensitic 440 and 420 stainless steels or Ti₆AI₄V titanium alloy. The pegs preferably have a pyramidal pointed shaped, for example with an equilateral triangle cross section. The longitudinal axes of the pegs are parallel to one another and perpendicular to the surface of an anterior one of the faces on the underside of the implant. Thus the pegs are inclined in a posterior/inferior direction and are perpendicular to the anterior surface of the resected talus. In a preferred example, at least one face of the pegs, such as a posterior-facing side of a peg having an equilateral triangle cross section, or both the anterior and posterior faces of a pyramidal peg having a square cross section, is oriented perpendicular to the sagittal plane and in place to oppose forces arising during flexing of the ankle.

As so structured, the peg is readily driven into the resected talus, forming a complementary opening at which the bone tissue of the talus is compressed against the peg. In one embodiment, at least the edges at which the faces of the peg meat, and alternatively or additionally the faces themselves, are serrated in the integral peg structure, to further secure the talar dome.

The disclosed implant is compliant in all aspects with ankle arthroplasty fixtures including preoperative navigation alignment guides for effecting resection of the talus and tibia, boring certain holes for receiving posts of intramedullary or other characters, and by which bone surfaces of the ankle are resected accurately to receive a tibial plate and talar dome.

The talar dome element can be affixed to the talus in a surgical step comprising anterior insertion of the talar dome element into position anterior of its final position (according to the cosine of the angles of the anterior face and the peg), and driving the talar dome by one or more impacts applied toward the talus in a direction parallel to the longitudinal axis of the pegs. This sets the pegs into talus and brings the surfaces of the talar dome into surface abutment with the resected surfaces of the talus (preferably with a layer of bone cement).

### BRIEF DESCRIPTION OF THE DRAWINGS

These and other objects and aspects will be appreciated by the following discussion of preferred embodiments and examples, with reference to the accompanying drawings, and wherein: FIG. 1 is a perspective illustration of a total ankle replacement prosthesis according to the present disclosure.

FIG. 2 is a perspective illustration of the talar dome element of the total ankle replacement prosthesis shown in FIG. 1.

FIG. 3 is a medial side elevation showing a human talus, marked for resection.

FIG. 4 is an anterior side elevation of the human talus as resected.

FIG. 5 is a schematic illustration, partly in section, showing the relationship of the prosthetic talar dome to the resected talus.

FIG. 6 is a side elevation of the talar dome prosthesis, with an inset showing the shape and orientation of one of the affixation pegs of the prosthesis.

FIG. 7 is a schematic illustration of setting the talar dome prosthesis into the talus.

FIG. 8 is a medial side elevation of the installed talar dome prosthesis.

FIG. 9 is a series of perspectives showing several advantageous shapes for the affixation pegs.

FIG. 10 is a schematic illustration with an inset detail, showing serrations at an edge between adjacent sides of an affixation peg.

FIG. 11 is a perspective showing serration lines on the sides of the affixation peg.

### DETAILED DESCRIPTION OF EXEMPLARY EMBODIMENTS

FIG. 1 is a perspective illustration of a total ankle replacement prosthesis including a talar dome prosthetic element 22 according to the present disclosure. The total ankle replacement prosthesis includes a tibial prosthesis 24 that articulates with the talar dome prosthesis 22. The talar and tibial prostheses 22, 24 slide over one another along arched interfacing surfaces 25, 27. Surfaces 25, 27 complement one another, each following an arch or curve around a horizontal lateral center line. The articulation of the prosthetic tibial and talar prostheses 24, 22 approximates the articulation between a natural talus and the tibiofibular joint or syndesmosis. The tibiofibular joint functions as a mortise for the talar dome as a tenon, permitting ranges of angular displacement. A primary displacement is dorsi-flexion/planter-flexion (relative rotation on a lateral horizontal axis of rotation) wherein the tibia and the foot can be inclined anteriorly and posteriorly relative to one another during gait. Additionally, inversion/eversion is a limited displacement in which the foot and tibia are rotated laterally inwardly or outwardly (on a medial horizontal axis) at the ankle. Abduction/adduction is a displacement wherein the foot is aligned laterally or medially relative to a sagittal plane (vertical axis of rotation). It is an object of the prosthetic ankle to approximate the degree of freedom of displacement that is characteristic of the natural ankle joint.

The talar dome prosthesis 22 can be an integral forging of surgical steel, shaped as shown and polished on its articulating surface 25. The tibial prosthesis 24 comprises a tibial plate 26 and a wear element 28 received therein. The wear element can comprise a high density polyethylene or similar material capable of withstanding carrying the patient's weight and sliding smoothly over the talar dome over a long useful life.

The tibial prosthesis 24 and the talar dome need to be permanently and rigidly affixed to the tibia and the talus, respectively. US 2012/0271314-Stemniski et al., which has been incorporated by reference, teaches techniques for resecting a tibiofibular joint to receive a tibial plate and resecting the talus to receive a talar dome prosthesis, both guided using the same navigation and guidance fixture for controlling the paths of surgical saws, drills and reamers applied from the anterior and plantar sides. In the Stemniski technique, the attachments to the tibia include providing an intramedullar bore in the tibia for receiving an elongated shaft element. In the present embodiment as shown in FIG. 1, the tibial plate carries three anchoring pins 32 that are embedded in the bores drilled and reamed in the resected tibia to securely affix the tibial plate 26 and thereby to securely fix the tibial prosthesis to the distal tibia. The tibial plate can carry a sintered porous metal surface (not shown) to enhance the attachment by encouraging bone ingrowth.

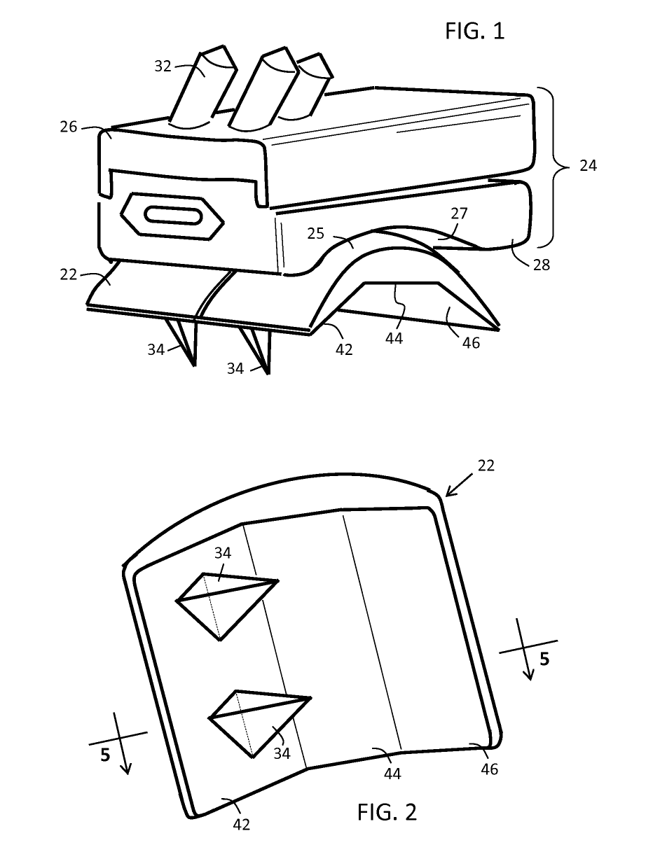

It is an aspect of the present invention that the talar dome prosthesis 22 is attached to a resected talus by virtue of complementary surface shapes together with a plurality of pegs 34 that are embedded in the resected talus to secure the talar dome 22. As shown in FIGS. 1 and 2, the talar dome prosthesis 22 consists essentially of an integral body of material, especially surgical steel or titanium alloy. On the underside facing the talus, the talar dome prosthesis 22 has a plurality of shaped surfaces 42, 44, 46 that are arranged to complement that shape of the talus after resection. A plurality of pegs 34 extend from the surface and are embedded in the talus to hold the talar dome prosthesis 22 securely in place when installed. In this embodiment the pegs are integral with the dome body and have a polygonal cross section tapering to a distal point.

The total ankle replacement (ankle arthroplasty) prosthesis comprises a talar dome prosthesis 22 configured for affixation to a talus bone. The talar dome 22 has a dome body with an articulating side 25 (the top side in FIG. 1) for bearing toward the tibia and a mounting side (shown in FIG. 2) for attachment to the talus bone, namely the underside. The mounting side has plural flat sections, the anterior one of which flat sections carries at least two pegs 34 rigidly protruding from the dome body. The pegs 34 are each tapered to a point for embedment in the talus bone for operatively attaching the talar dome to the talus bone.

The mounting side of the talar dome 22 as shown in FIG. 2 has three flat surfaces 42, 44, 46 that are angularly inclined relative to one another. The central flat surface 44 is aligned substantially horizontal and rests on the top surface of a talus that has been re-sected by being sawn off horizontally. The anterior and posterior surfaces 42, 46 are inclined inferiorly from the horizontal central surface 44, namely downwardly toward the front and rear edges, respectively, and rest on complementary resected flat surfaces of the talus. The structure forms the underside of the talar dome prosthesis 22 into a faceted female cup shape that fits in surface contact with the faceted resected talus. Due to vertically downward pressure from the weight of the patient on the ankle, the talar dome is held against the talus. The inclined anterior and posterior facet surfaces 42, 46 contribute to holding the talar dome 22 in place on the talus and the pegs 34 further maintain the position of the talar dome 22.

In the embodiment shown, the pegs 34 extend perpendicularly from the anterior flat section. As a result, the pegs extend are obliquely inclined relative to horizontal, downwardly and toward the rear of the talus bone. With a nominal gait, pushing off with some degree of dorsi-flexion applies the patient's weight in a direction more or less parallel to the longitudinal axes of the pegs 34. Stepping forward into plantar-flexion causes the wear element 28 to slide anteriorly over the talar dome sliding surface 25 in a direction corresponding to the insertion direction of pegs 34, which is partly posteriorly.

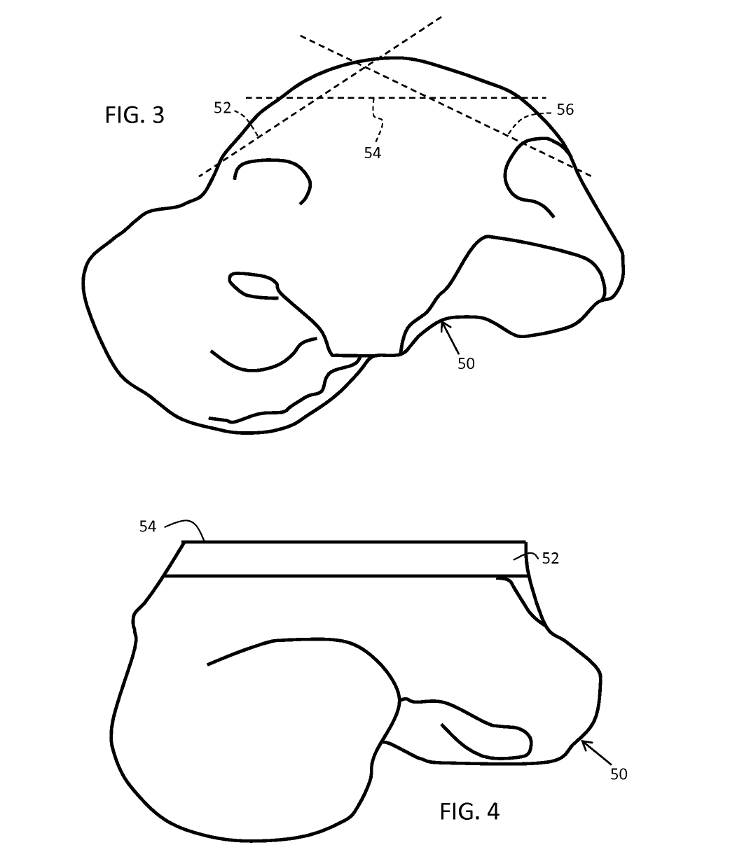

For providing complementary surfaces on the talus and the talar dome 22, the talus is resected during the surgical process ankle arthroplasty. In the medial side elevation view of FIG. 3, a human talus 50 is shown with planes marked for resection. Three saw cuts are made, preferably guided by an alignment and navigation fixture with appropriate guides for the angle and transit of the surgical saw, along the lines 52, 54 and 56. These lines define planes at angles that complement the angles of surfaces 42, 44, 46 on the underside of the talar dome prosthesis 22. Each cut removes the bone tissue superior to the cut line, leaving a resected talus as shown in an anterior side elevation view in FIG. 4, where the bone surfaces 52 and 54 are seen obliquely or edge-on. Whereas the saw cuts are controlled by the alignment/navigation feature to precisely match the dimensions and angles of surfaces 42, 44, 46 of talar dome 22, the talar dome fits precisely on the talus, but for the pointed pegs 34.

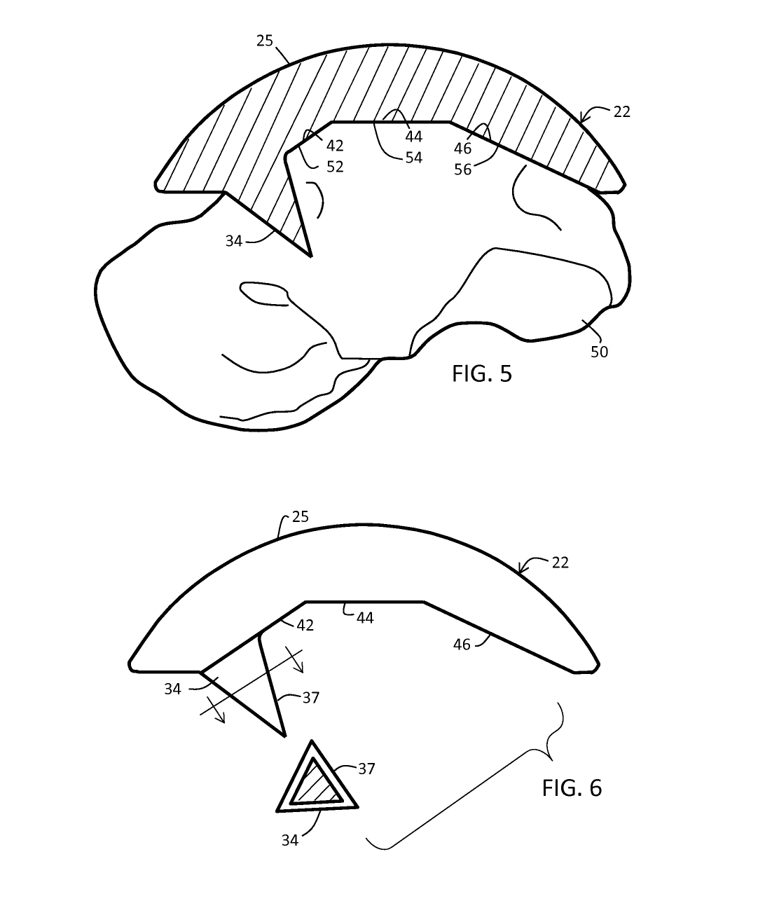

FIG. 5 is a schematic illustration, the talar dome shown in section along line 5-5 in FIG. 2. This figure shows surfaces 42, 44, 46 of the talar dome prosthesis 22 abutting directly against resected surfaces 52, 54, 56 of talus 50. The surface contact is such that the peg 34 is fully embedded in talus 50, up to the inclined surface 42 from which peg 34 protrudes.

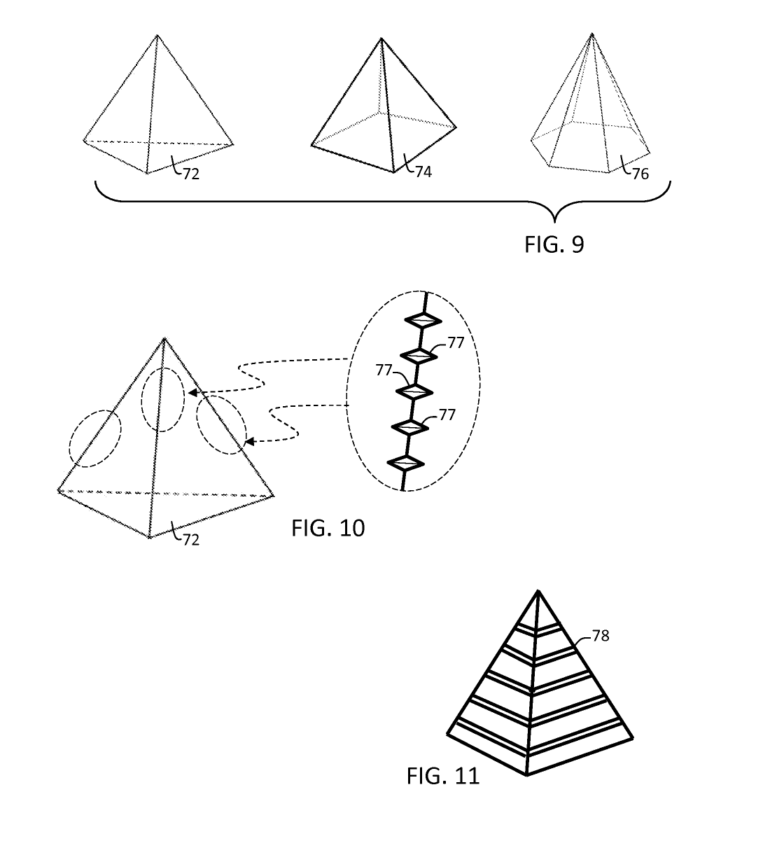

FIG. 6 is a side elevation of the talar dome prosthesis 22, with an inset showing the shape and orientation of one of the affixation pegs 34 of the prosthesis 22. In this embodiment the peg 34 is triangular in cross section as shown, tapering on three sides to a point. In this embodiment the peg 34 is a regular tetrahedron. Alternative shapes are possible, some being shown in FIGS. 9-11 including other tapered pegs with polygonal cross section. An advantageous aspect of a polygonal cross section, including the embodiment in FIG. 6, is that the posteriorly facing side 37 of the peg 34 is perpendicular to the sagittal plane. This orientation provides maximum opposition to forces arising parallel to the sagittal plane in walking. In FIG. 6, the triangular cross section defines a posterior flat side and an anterior edge. In other alternatives shown in FIG. 9, both the anterior and posterior sides can be perpendicular to the sagittal plane, e.g., in a peg shaped as a pyramid with a square bottom or in other polygons with an even number of sides (four, six, eight, etc.). The triangular cross section of a tetrahedron or the square cross section of a square bottom pyramid are the least complicated to manufacture.

For manufacturing, an integral talar dome element is provided in the general shape shown, for example cast in one piece. The bottom surfaces can be machine to flat precision at the necessary angles. The sliding upper surface 25 is polished. The sides of pegs 34 are polished. A porous coating such as sintered titanium alloy particles as in Wright Medical Technology BIOFOAM® (not shown) can be applied to surfaces 42, 44, 46 to improve prospects for bone ingrowth. It would be possible to likewise apply a porous coating to the peg 34, but in general a smooth peg is more readily driven into the talus 50 than a peg thickened by a porous coating.

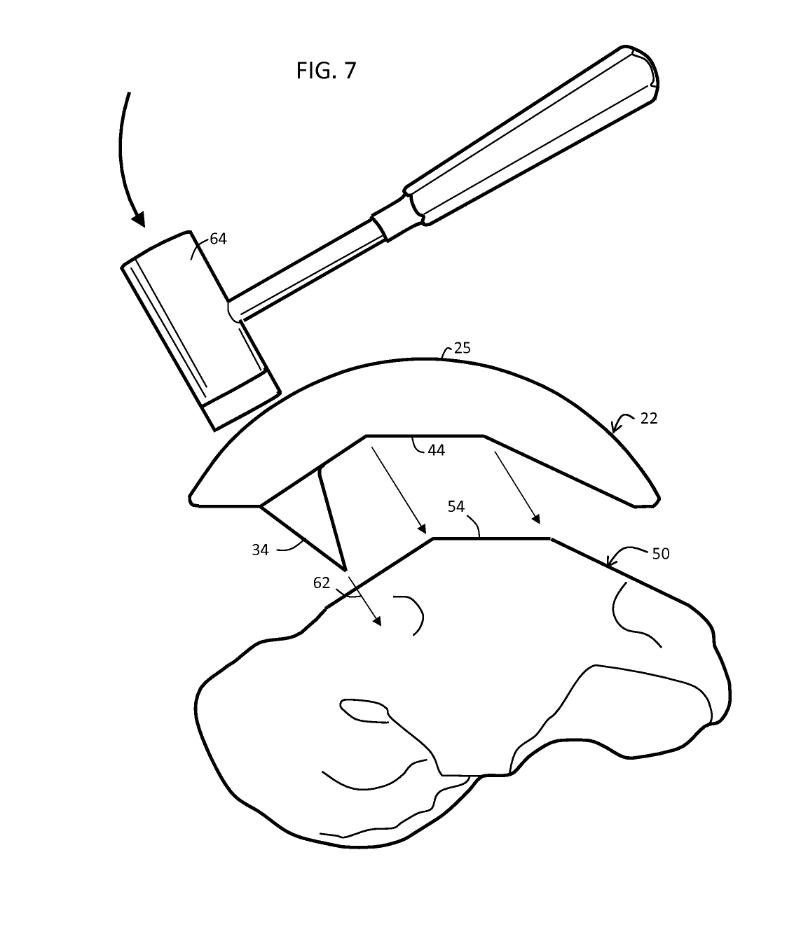

FIG. 7 is a schematic illustration showing driving the talar dome prosthesis 22 into the talus 50 such that the respective faces 42 etc. of the prosthesis and 52 etc. of the talus are brought into surface abutment. A pilot hole can be drilled at line 62 to predetermine the point of entry. The talar dome prosthesis 22 is positioned anteriorly of its final position by a distance that accounts for the posterior/inferior orientation of the peg 34. Preferably, forming the pilot hole at line 62 and the placement and orientation of the talar dome prosthesis are determined by the alignment and navigation fixture.

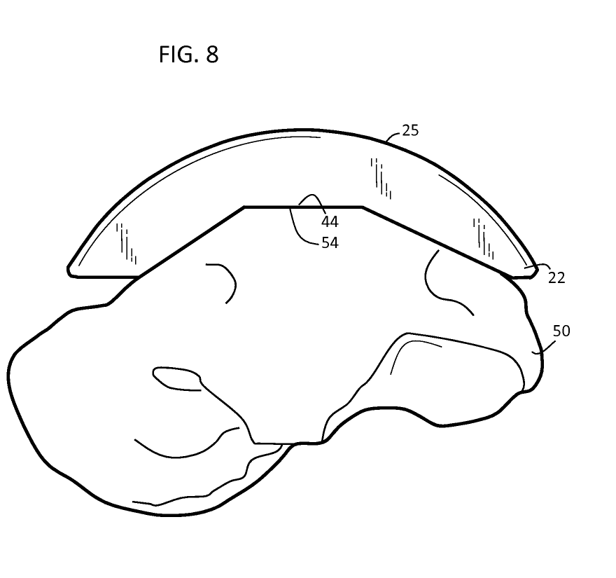

The prosthesis 22 is driven home with a mallet 64 or other similar tool, which preferably is faced with a polymer material so as not to mar the sliding surface 25. This places the talar dome prosthesis 22 in its final position shown in FIG. 8. It may be noted that the underside of prosthesis 22 conforms exactly to the facing surfaces of the resected talus. The prosthesis preferably resides exclusively on top of the talus 50, wrapping over the anterior and posterior but not having lateral and/or medial side flanges depending downwardly. Omitting the side flanges provides for clear fluoroscopic visualization and avoids any need to trim the lateral and medial sides of the talus to accommodate prosthesis 22.

In FIG. 6, the pegs 34 form tetrahedral pyramids with a triangular cross section and triangular base forming an equilateral triangle. The pegs extend substantially perpendicularly from flat section 42 on the mounting side of the prosthetic dome body 22. Variations are possible in peg shape and orientation. For example, the pegs can be on an axis that is inclined relative to surface 42, the prosthesis 22 being driven into place on a line parallel to the peg axis instead of perpendicular to surface 42.

In FIG. 9, a series of perspective phantom illustrations show advantageous shapes for the affixation pegs 34, including a tetrahedron 72, a square base pyramid 74 and a polygonal structure with an even number of sides, for example a hexagonal pyramid 76. As previously noted, one of the sides is preferably oriented on the posterior side perpendicular to the sagittal plane. The peg shapes are shown separately in FIG. 9, and could comprise one or more parts that are attached to surface 42 of prosthesis 22. But preferably pegs 34 are formed integrally in one piece with the remainder of prosthesis 22 from their bases to their pointed tips. The center axes of the pegs are oriented perpendicular to the plane of surface 42.

As shown in FIG. 10, the pegs are not required to be entirely smooth. A trapezoidal peg 72 in FIG. 10 is provided with serrations 77 along the edges at which the faces meet. The serrations are useful to lock the prosthesis in place after a period of healing, due to ingrowth of bone tissue into serrations 77. In an alternative embodiment in FIG. 11, the faces of a peg are shown with grooves 78, forming an alternative type of serration that likewise locks the prosthesis 22 in place after a period of bone ingrowth.

The pegs are capable of embodiment in shapes other than polyhedrons, especially pyramids with sides that meet at three or four edges, at least one being perpendicular to the sagittal plane. In the depicted embodiment, the pegs are obliquely inclined relative to horizontal, downwardly and toward a rear of the talus bone, because the longitudinal axes of the pegs are perpendicular to the plane defined by the anterior flat section 42, which is inclined downwardly (inferiorly) toward the anterior edge of the talar dome prosthesis.

Flat section or face 42 is one of a plurality of flat faces on the underside of the dome body, configured to abut against a resected surface of the talus having a shape that is complementary with the underside, namely cut or machined away to define the same sequence of flat faces. The underside of the dome body is fully defined by the flat faces. That is, the prosthesis lacks depending lateral and medial flanges, instead wrapping over the top of the resected talus and defining the smooth and rounded articulating upper surface in an arc over the talus, for bearing against the tibial prosthesis. The talar dome is formed as an one-piece part with the dome body and pegs being integral with one another, being readily manufactured, robust and structurally uncomplicated.

The invention has been disclosed in connection with a number of variations presented as examples. However the invention is not limited to the exemplary embodiments and is capable of additional variations. Reference should be made to the appended claims instead of the foregoing description, to assess the scope of exclusive rights in the invention claimed.

What is claimed is:

1. A prosthesis for ankle arthroplasty, comprising:
a talar dome configured for affixation to a talus bone, the talar dome having dome body with an articulating side for bearing toward a tibia and a mounting side for attachment to the talus bone;
wherein the mounting side includes at least two pegs rigidly protruding from an underside of the dome body forming the mounting side, the pegs each being tapered to a point for embedment in a surface of the talus bone for operatively attaching the talar dome to the talus bone.

2. The prosthesis of claim 1, wherein the pegs are integral with the dome body and have a polygonal cross section tapering to a distal point.

3. The prosthesis of claim 2, wherein the pegs have a triangular cross section.

4. The prosthesis of claim 2, wherein edges of the polygonal cross section are serrated.

5. The prosthesis of claim 1, wherein the pegs are shaped as pyramids each having a base with three edges.

6. The prosthesis of claim 5, wherein the pyramids each have a base forming an equilateral triangle and extend substantially perpendicularly from a flat section of the mounting side of the dome body.

7. The prosthesis of claim 6, wherein the flat section from which the pegs extend is one of plural flat sections that engage directly against machined faces of the talus bone that correspond after resection to the three flat sections.

8. The prosthesis of claim 7, wherein the flat section from which the pegs extend is arranged such that the pyramids of the pegs are obliquely inclined relative to horizontal, downwardly and toward a rear of the talus bone.

9. The prosthesis of claim 1, wherein the dome body defines a plurality of flat faces on the underside, configured to abut against a resected surface of the talus having a shape that is complementary with the underside.

10. The prosthesis of claim 9, wherein an anterior one of the flat faces is inclined upwardly from an inferior front edge to a central one of the flat faces, and wherein the pegs are disposed on said anterior one of the flat faces.

11. The prosthesis of claim 10, wherein the pegs have longitudinal axes perpendicular to said anterior one of the flat faces.

12. The prosthesis of claim 10, wherein a posterior one of the flat faces is inclined downwardly from the central one of the flat faces to an inferior rear edge.

13. The prosthesis of claim 9, wherein the underside of the dome body is defined by the flat faces, whereby the prosthesis lacks depending lateral and medial flanges.

14. The prosthesis of claim 1, wherein the talar dome is formed as an one-piece part with the dome body and pegs being integral with one another.
